(19) United States
(12) Patent Application Publication
(10) Pub. No.: US 2023/0210380 A1
(43) Pub. Date: Jul. 6, 2023

(54) TEMPERATURE DETECTION SYSTEM

(71) Applicants: EV-TECHNOLOGIES, COLOMBELLES (FR); CENTRE NATIONAL DE LA RECHERCHE SCIENTIFIQUE, PARIS (FR)

(72) Inventors: Sidina WANE, CAEN (FR); Azzedine BOUSSEKSOU, TOULOUSE (FR); Lionel SALMON, VILLEGAILHENC (FR); Gabor MOLNAR, TOULOUSE (FR); Quang-Hung TRAN, MONTPELLIER (FR)

(21) Appl. No.: 17/999,829

(22) PCT Filed: May 31, 2021
(86) PCT No.: PCT/EP2021/064510

§ 371 Date: Nov. 23, 2022

(30) Foreign Application Priority Data
May 29, 2020 (FR) 2005715

(51) Int. Cl. A61B 5/01; A61B 5/1455; A61B 5/00

(52) U.S. Cl. CPC A61B 5/01; A61B 5/681; A61B 5/14552; A61B 2562/0233

(57) ABSTRACT

A temperature detection system (10) comprising a layer of spin cross-over material (11) in thermal contact with a target surface (12); at least one first light source (13) configured to provide a first and a second illumination (13a, 33a) of at least a first portion (12a) of the layer of spin cross-over material (11); at least one first light receiver (14) configured to capture first and second return light (14b, 34b) coming from the layer of spin cross-over material (11) and resulting respectively from the first and second illuminations; generate a first signal (S1) based on the first return light (14b); and generate a second signal (S2) based on the second return light (34b); a computation circuit (15, 17) configured to determine, based at least on a correlation between the first and second signals (S1, S2), a temperature of the layer of spin cross-over material (11).

The present patent application claims priority from the French patent application filed on 29 May 2020 and assigned application no. FR20/05715, the contents of which is hereby incorporated by reference.

### TECHNICAL FIELD

The present disclosure relates generally to the field of temperature detection devices.

### BACKGROUND ART

Temperature detection devices for detecting the temperature of a target surface, such as the surface of an integrated circuit or the surface of the skin of a user, have been proposed. However, existing solutions tend to have relatively low accuracy, and/or are costly and/or unreliable and do not provide a precise measurement of the temperature.

Devices capable of detecting blood oxygen levels through the skin of a user have also been proposed, but it is challenging to provide a detection device capable of providing coherent detection of both temperature and blood oxygen.

### SUMMARY OF INVENTION

There is a need in the art for a temperature detection device that at least partially addresses one or more drawbacks in the prior art.

There is a supplementary need for a blood oxygen level measurement device that is accurate and is capable of providing coherent measurements with temperature measurements made by the temperature detection device.

Solutions are described in the following description, in the appended set of claims, and in the accompanying drawings.

One embodiment addresses all or some of the drawbacks of known temperature detection systems.

One embodiment provides a temperature detection system comprising:
a layer of spin cross-over material in thermal contact with a target surface;
at least one first light source configured to provide a first and a second illumination of at least a first portion of the layer of spin cross-over material;
at least one first light receiver configured to:
   capture first and second return light coming from the layer of spin cross-over material and resulting respectively from the first and second illuminations;
   generate a first signal based on the first return light; and
   generate a second signal based on the second return light;
a computation circuit configured to determine, based at least on a correlation between the first and second signals, a temperature of the layer of spin cross-over material.

According to one embodiment, the first and second signals are generated according to a first characteristic of respectively the first and second return lights which is relative to a reflectivity and/or a color of the layer of spin cross-over material.

According to one embodiment, the first characteristic is an optical intensity.

According to one embodiment, the spin cross-over material comprises, and for example consists of, [Fe(HB(1,2,4-triazol-1-yl)₃)₂]bis[hydrotris(1,2,4-triazol-1-yl)borate]Fe(II).

According to one embodiment, a layer of a temperature conductive material is arranged between the target surface and the layer of spin cross-over material.

According to one embodiment, the first and second illuminations are each formed of light having a same first wavelength.

According to one embodiment, the computation circuit is configured to determine the correlation between the first and second signals using an auto-correlation calculation or a cross-correlation calculation, the auto-correlation calculation for example being performed when the first and second signals are issued from a same portion of the layer of spin cross-over material and the cross-correlation calculation for example being performed when the first and second signals are issued from different portions of the layer of spin cross-over material, the different portions for example being adjacent portions, the auto-correlation or cross-correlation calculation for example being approximated by a Cardinal-Sine function.

According to one embodiment, the computation circuit is configured to determine the temperature of the layer of spin cross-over material further based on one or more reference values associated with known temperatures.

According to one embodiment, the temperature detection system is further comprising at least one second light source configured to provide a third and a fourth illumination of at least the first portion of the layer of spin cross-over material, wherein the first and second illuminations are each formed of light having a same first wavelength and the third and fourth illuminations are each formed of light having a same second wavelength different to the first wavelength; the first light receiver being configured to:
capture third and fourth return light coming from the layer of spin cross-over material and resulting respectively from the third and fourth illuminations;
generate a third signal based on the third return light; and
generate a fourth signal based on the fourth return light; the third and fourth signals being generated according to the optical intensity of respectively the third and fourth return lights;
the computation circuit being configured to determine the temperature of the layer of spin cross-over material further based on a correlation between the third and fourth signals.

According to one embodiment, the temperature detection system is further comprising:
at least one third light source configured to provide a fifth and sixth illuminations of at least a second portion of the layer of spin cross-over material, the fifth and sixth illuminations being formed of light having a third wavelength substantially equal to the first wavelength;
at least one fourth light source configured to provide a seventh and eighth illuminations of at least the second portion of the layer of spin cross-over material, the seventh and eighth illuminations being formed of light having a fourth wavelength substantially equal to the second wavelength;
a second first light receiver being configured to:
   capture fifth, sixth, seventh and eighth return light coming from the layer of spin cross-over material and resulting respectively from the fifth, sixth and seventh and eighth illuminations;
   generate a fifth signal based on the fifth return light;
   generate a sixth signal based on the sixth return light;
   generate a seventh signal based on the seventh return light; and
   generate an eighth signal based on the eighth return light;
the fifth, sixth, seventh and eighth signals being generated according to the optical intensity of respectively the fifth, sixth, seventh and eighth return light;
the temperature detection system comprising a third correlator configured to run a third operation of correlation, between the fifth and sixth signals;
the third operation of correlation consisting in an autocorrelation operation and/or a cross-correlation operation;
the temperature detection system comprising a fourth correlator configured to run a fourth operation of correlation, between the seventh and eighth signals;
the fourth operation of correlation consisting in an autocorrelation operation and/or a cross-correlation operation;
the analyzer being arranged to determine the temperature of the layer of spin cross-over material at the second portion using a result of the third and fourth operations of correlation and the first reference.

According to one embodiment, the at least one first and second light sources are each configured to provide the first, second, third and fourth illuminations to illuminate sequentially, portions of the layer of spin cross-over material;
the first light receiver being configured to:
   generate the first signal for each portion of the layer of spin cross-over material sequentially illuminated by the first illumination;
   generate the second signal for each portion of the layer of spin cross-over material sequentially illuminated by the second illumination;
   generate the third signal for each portion of the layer of spin cross-over material sequentially illuminated by the third illumination;
   generate the fourth signal for each portion of the layer of spin cross-over material sequentially illuminated by the fourth illumination;
the second light receiver being configured to:
   generate the fifth signal for each portion of the layer of spin cross-over material sequentially illuminated by the fifth illumination;
   generate the sixth signal for each portion of the layer of spin cross-over material sequentially illuminated by the sixth illumination;
generate the seventh signal for each portion of the layer of spin cross-over material sequentially illuminated by the seventh illumination;
   generate the eighth signal for each portion of the layer of spin cross-over material sequentially illuminated by the eighth illumination;
the first, second, third, fourth, fifth, sixth, seventh and eighth signals being generated according to the optical intensity of the respective return lights.

According to one embodiment, the first light source and/or the second light source and/or the third light source and/or the fourth light source are mobile in relation to the layer of spin cross-over material.

According to one embodiment, the first light source, the second light source and the first light receiver are fixed in relation to each other and mobile around a first axis; and/or the third light source and the fourth light source and the second light receiver are fixed in relation to each other and mobile around a second axis.

According to one embodiment,
the first and/or the second light source comprises a plurality of light emitters, for example 4, 6 or 8 light emitters, configured to illuminate a plurality of different portions of the layer of spin cross-over material; and/or
the at least one first or second light receiver comprising a plurality of light detectors, for example 4, 6 or 8 light detectors, configured to receive return light from a plurality of different portions of the layer of spin cross-over material.

According to one embodiment, the plurality of light emitters and/or the plurality of light detectors are arranged in a linear or 2-dimentional matrix of pixels.

According to one embodiment:
the layer of spin cross-over material is arranged to allow at least part of the first, second, third and fourth illuminations to propagate through it and reflects on an interface of the layer of spin cross-over material facing the target surface resulting respectively in a first, second, third and fourth target return light;
the first light receiver being further configured to:
   generate a first target signal based on the first target return light;
      generate a second target signal based on the second target return light;
      generate a third target signal based on the third target return light;
      generate a fourth target signal based on the fourth target return light;
the first, second, third and fourth target signals being generated according to the optical intensity of respectively the first, second, third and fourth target return light;
wherein the computation circuit is further configured to:
   determine a correlation between the first and second target return signals;
   determine a correlation between the third and fourth target return signals; and
   the computation circuit being capable of determining, based on the correlations between the first and second target return signals and between the third and fourth target return signals and on a Stern-Volmer constant, a blood oxygen level at the target surface.

According to one embodiment, the target surface is a surface of a device under test, such as one or more transistors or one or more integrated circuit chips; a surface of a vehicle; a surface of an animal or a surface of a human body.

According to one embodiment, the temperature detection system is further comprising an energy harvesting device configured to harvest heat energy from the target surface to power components of the temperature detection system.

According to one embodiment, at least the first light source and the first light receiver are formed together in a same photodiode.

According to a further aspect, there is provided a temperature detection device comprising the temperature detection system.

According to yet a further aspect, a bracelet is comprising the temperature detection system.

According to one aspect, the bracelet is comprising the energy harvesting device, and the target surface is the skin of a user of the bracelet, for example in the wrist or ankle region, and energy is harvested based on a temperature gradient between the skin temperature and an ambient air temperature.

According to one embodiment, there is provided a temperature detection device comprising:
a layer of smart-spin material;
at least one light source configured to illuminate the layer of smart-spin material; and
at least one photodiode configured to capture return light from the layer of smart-spin material.

According to one embodiment, the at least one light source is at least one light-emitting diode.

According to one embodiment, the at least one photodiode comprises at least a first photodiode arranged to be sensitive to light of a first range of wavelengths, and at least a second photodiode arranged to be sensitive to light of a second range of wavelengths different to the first range.

According to one embodiment, the temperature detection device is further comprising a processing device configured to determine, based on an output signal of the at least one photodiode, an output color signal comprising one or more digital values representing a color of the layer of smart-spin material.

According to one embodiment, the processing device is further configured to convert the output color signal into a temperature value.

According to one embodiment, the temperature value has an accuracy of 0.05° C. or better.

According to one embodiment, the processing device is configured to perform said conversion by comparing the one or more digital values of the output color signal with one or more thresholds.

According to one embodiment, the temperature detection device is further comprising a time-of-flight ranging device configured to determine a distance separating the target surface from one or more regions of the layer of smart-spin material.

According to one embodiment, the time-of-flight ranging device is configured to detect a time-of-flight of an illumination pulse generated by the at least one light source, the layer of smart-spin material being configured to allow at least some of the light of the illumination pulse to propagate through it.

According to one embodiment, the at least one light source comprises a plurality of light sources, for example 4, 6 or 8 light sources, configured to illuminate a plurality of different regions of the layer of smart-spin material, and/or the at least one photodiode comprising a plurality of photodiodes, for example 4, 6 or 8 photodiodes, configured to receive reflected light from a plurality of different regions of the layer of smart-spin material.

According to one embodiment, the time-of-flight ranging device is configured to determine the distance separating each of said different regions from the target surface, and to detect the region closest to the target surface, wherein one or more of the light sources and/or one or more of the photodiodes is selected to perform the temperature detection in the closest region.

According to one embodiment, the temperature detection device is further comprising an energy harvesting device configured to harvest heat energy from a target surface.

According to one embodiment, the target surface is the surface of a device under test (DUT), such as one or more transistors or one or more integrated circuit chips.

According to a further aspect, there is provided a bracelet comprising the temperature detection device.

According to an aspect, the target surface is the skin of a user of the bracelet, for example in the wrist or ankle region, and energy is harvested based on a temperature gradient between the skin temperature and an ambient air temperature.

### BRIEF DESCRIPTION OF DRAWINGS

The foregoing features and advantages, as well as others, will be described in detail in the following description of specific embodiments given by way of illustration and not limitation with reference to the accompanying drawings, in which:

FIG. 1 schematically illustrates a temperature detection device according to an embodiment of the present disclosure;

FIG. 2 is a graph representing reference curves used by embodiments of the present disclosure;

FIG. 3 schematically illustrates a temperature detection device according to a further embodiment of the present disclosure;

FIG. 4 schematically illustrates a temperature detection device according to a further embodiment of the present disclosure;

FIG. 5 schematically illustrates a temperature detection device according to yet a further embodiment of the present disclosure;

FIG. 6 schematically illustrates a temperature detection device having scanning capabilities according to an embodiment of the present disclosure;

FIG. 7 schematically illustrates a temperature detection device having scanning capabilities according to a further embodiment of the present disclosure;

FIG. 8 illustrates a temperature detection device in the form of a bracelet according to an embodiment of the present disclosure; and

FIG. 9 schematically illustrates a test system according to an embodiment of the present disclosure.

### DESCRIPTION OF EMBODIMENTS

Like features have been designated by like references in the various figures. In particular, the structural and/or functional features that are common among the various embodiments may have the same references and may dispose identical structural, dimensional and material properties.

For the sake of clarity, only the operations and elements that are useful for an understanding of the embodiments described herein have been illustrated and described in detail.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following disclosure, unless indicated otherwise, when reference is made to absolute positional qualifiers, such as the terms “front”, “back”, “top”, “bottom”, “left”, “right”, etc., or to relative positional qualifiers, such as the terms “above”, “below”, “higher”, “lower”, etc., or to qualifiers of orientation, such as “horizontal”, “vertical”, etc., reference is made to the orientation shown in the figures, or to the orientation of the temperature detection device during normal use.

Unless specified otherwise, the expressions “around”, “approximately”, “substantially” and “in the order of” signify within 10%, and preferably within 5%.

This disclosure relates to a temperature detection system for detecting a temperature of a target surface. In the present disclosures the term “target surface” refers either to an external face of the target to be measured, or to a region close to the external face and extending internally for example by few millimeters or few centimeters.

A temperature measurement of a target surface is usually made by infrared measurement. However, the methods available are not precise and a micron scale measurement is not available at acceptable cost and speed of acquisition.

The blood oxygen levels at the surface of the skin of human or animal body can be measured by a portable equipment. Unfortunately, the system employed to measure blood oxygen levels using such equipment is different from the system based on infrared sensing used for the temperature measurement. The measurement is thus taken at different locations, and as such it is not possible to obtain coherent temperature and blood oxygen readings using existing solutions.

The temperature detection system disclosed takes advantage of a spin cross-over material (SCO) applied on, or otherwise in thermal contact with, a target surface to determine indirectly a parameter of the target surface, from which the temperature can be deduced.

The SCO is a material having a known response to a light illumination as a function of an inner parameter, such as its temperature. For example, the optical response to a light illumination of the SCO will change drastically as a function of the temperature of the SCO. The SCO is for example deposited as a relatively thin layer, and thus the temperature of the SCO rapidly corresponds to the temperature of the target surface. Thus, by determining the temperature of the SCO layer it is possible to obtain a relatively precise determination of the temperature of the underlying target surface. Such a system is interesting because the optical response of the SCO varies strongly as a function of its temperature. A precise determination of the temperature is thus obtained. The SCO may be easily fabricated at low cost and applied on a target surface even if the target surface is 3-dimensional. The present disclosure allows therefore a precise and cost-effective surface temperature detection of a target surface.

The SCO layer, as applied on or otherwise in thermal contact with the target surface, is also compatible with a direct determination of other parameters of the target surface such as a blood oxygen level in the case that the target surface is the skin of a human or animal body. Indeed, the SCO layer for example allows light illumination to propagate through it and reach the target surface. It is therefore possible to determine coherent measurements of temperature and blood oxygen levels at the same precise region of the target surface.

Among the possibilities enabled by this disclosure are:
Battery-less operation (compliant with Low-Energy ASIC solution) using Thermal-harvesting for: the Energy-Harvesting resulting from the differential temperature between the sensed temperature of the DUT and the ambient temperature.
Nano-Thermometry scale imaging with real-time visualization of Temperature Gradients and Transient variations.
Temperature-controlled tuning of RF/mmWave and optical systems: True-Delay-Lines, Resonators, Fabry-Perrot, Light-Fidelity (LiFi), etc.
NV-based Near-Field and Far-Field Scanning of components, circuits and Systems.
Optical Beamforming/Beamsteering.

Among targeted applications are:
Smart-IoT: Wearables
5G/Beyond 5G
LiFi & Photonics
Industrial & R&D Instrumentation
Medical Applications

Smart Bracelet with Body Thermal-Sensor with the following features:
Wireless communication of measured body-temperature using LoRa & Bluetooth protocols.
Simultaneous Measurement of several peoples with data protection (security).
Thermal-Body Harvesting combined with rechargeable battery/supercapacitor.

VEP used for the following benefits:
Smart-Processing of sensors data real-time monitoring.
Smart Power-Management.
Aggregation and correlation analysis of several sensors:

EEG, Thermal, oxygen, etc.

Data security protection.
Use of Inductive-Charging combined with the sensors.

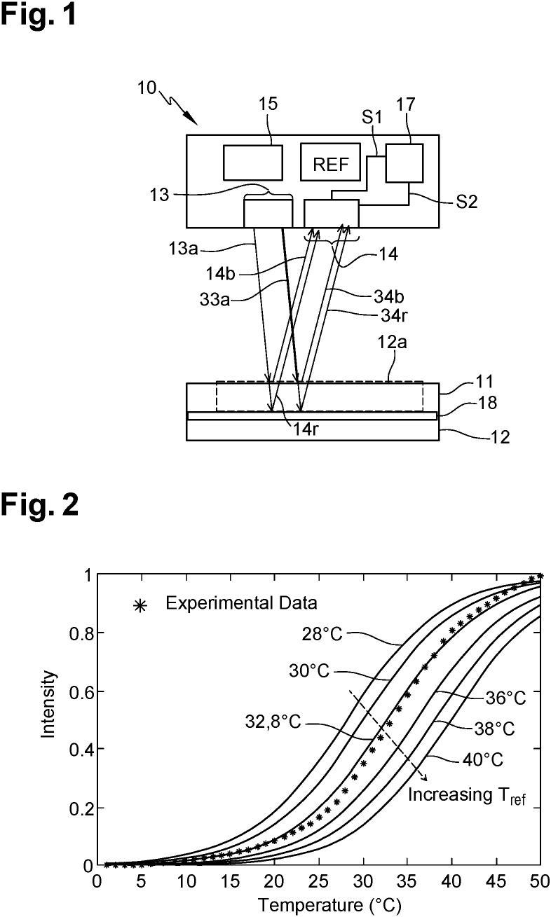

FIG. 1 illustrates a temperature detection system 10 according to an embodiment of the present disclosure.

The temperature detection system 10 of FIG. 1 comprises a layer 11 of spin cross-over material (SCO), at least one first light source 13, at least one first light receiver 14 and a computation circuit comprising an analyzer module 15 and a correlator 17.

The computation circuit, and in particular the analyzer module 15 and correlator 17, are for example implemented by a hardware circuit, such as by an application specific integrated circuit (ASIC) and/or by a field programmable gate array (FPGA). Alternatively, the functions of the computation circuit could be implemented at least partially in software, the computation circuit for example comprising a processor, such as a microprocessor or CPU (Central Processing Unit) configured to execute instructions that cause at least some of the functions of the computation circuit to be implemented.

The layer 11 of SCO is for example positioned in thermal contact with the target surface 12. In some embodiments, the layer of SCO is maintained directly in contact with the target surface 12 or at a close distance, typically at less than one millimeter away from the target surface 12. In other embodiments, the layer 11 is deposited as a coating on the target surface 12. In still other embodiments, the layer 11 is in thermal contact with the target surface 11 via one or more intermediate thermally conductive layers. In some embodiments, the layer 11 of SCO is close enough of the target surface that the temperature of the layer of SCO 11 follows that of the target surface 12 with a relatively very low time latency.

The SCO layer 11 for example has a thickness of between 50 nm and 1 mm for high transparency, for example 50 nm,100, 200, 500, or 900 nm and in some cases of between 1 µm and 500 µm for high sensitivity, for example 1, 20, 100, 200, or 500 µm.

In an example illustrated in FIG. 1, a layer 18 of thermally conductive material is arranged between the target surface 12 and the SCO 11. The layer 18 of thermally conductive material 18 is for example a relatively thin metal plate, which can be crenelated to ease the measurements. The metal plate for example has a thickness of between 100 µm and 1 mm.

In an example, the layer 11 of SCO is made of, or comprises, [Fe(HB(1,2,4-triazol-1-yl)₃)₂]bis[hydrotris(1,2,4-triazol-1-yl)borate]Fe(II). This material formula may also comprise additional H2O compounds.

For example, a spin cross-over material is described in the following publication: Olena Kraieva, Carlos Mario Quintero, Iurii Suleimanov, Edna Hernandez, Denis Lagrange, et al., “High Spatial Resolution Imaging of Transient Thermal Events Using Materials with Thermal Memory”. Small, Wiley-VCH Verlag, 2016, 12 (46), pp.6325-6331. 10.1002/smll.201601766. hal-01413097, the contents of this publication being hereby incorporated by reference.

In the case that the SCO layer 11 is formed by deposition, it is for example deposited directly on the target surface 12, or on the thermally conductive layer 18, by evaporation, chemical vapor deposition, spin coating, doctor blade coating, spraying or any other usual techniques known to a person skilled in the art.

In the example of FIG. 1, the target surface 12 is not part of the temperature detection system. The temperature detection system 10 is employed to determine at least the temperature of the target surface 12. The target surface 12 is for example the skin of a user, for example in the wrist or ankle region. It may also be a surface of a device under test (DUT), such as one or more transistors or one or more integrated circuit chips; a surface of a vehicle; a surface of an animal or a surface of a human body.

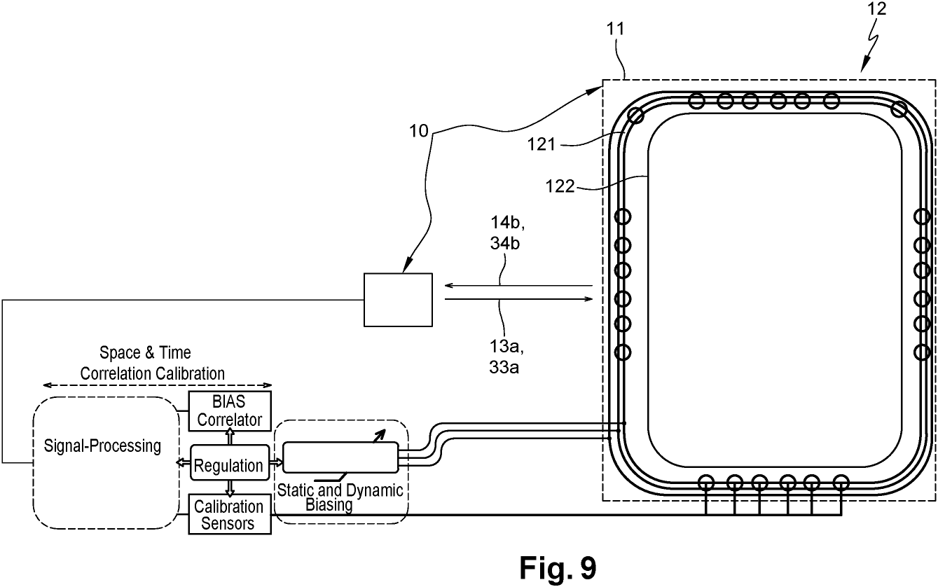

In the case where the layer 11 of SCO is deposited on a surface of a device to test (DUT), the layer 11 of SCO could then be part of the protective layer that is usually formed on the circuits or devices to protect them against humidity or mechanical shocks. For example, the layer of SCO could be embedded with a nitride. The DUT may be designed specially to allow a fast and precise determination of a heating spot or an electrical current mapping. Such an implementation will be described in relation with FIG. 9.

In the case where the layer 11 of SCO is positioned on a surface of a vehicle, the SCO may be part of an antenna film or a meta material film. This configuration provides an advantage for well recognized and well-hidden purposes.

The first light source 13 is configured to illuminate, with a first illumination 13a and a second illumination 33a, at least a first portion 12a of the layer of spin cross-over material 11.

The first and second illuminations 13a, 33a are for example emitted sequentially.

In an example, the first light source 13 is a light emitting diode, or a pair of light emitting diodes, or a linear array or 2-dimensional matrix of light emitting diodes.

In another example, the first light source 13 is a laser or an array of lasers, such as for example laser diodes or vertical-cavity surface-emitting lasers (VCSEL).

The first and second illuminations 13a, 33a are for example formed of light in the visible or infrared or ultraviolet wavelength range.

In an example, the first and second illuminations 13a, 33a are formed by the same, or substantially the same, first wavelength of light generated sequentially or simultaneously during, for example, a first period of time Ta, of a few microseconds, or a few tens or hundreds of microseconds. The first and second illuminations 13a, 33a are for example repeated in time.

The first or second illuminations 13a, 33a may, in an example, illuminate the SCO layer 11 with illumination pulses. In equivalent terms, the first light source 13 is for example configured to generate the first or second illuminations 13a in the form of pulses. This configuration may be employed in order to obtain time-of-flight information to be able to determine a distance of one or more points on the layer 11 of spin cross-over material.

In an example, the first light source may provide the first and second illuminations sequentially to further different portions of the SCO layer 11, until the entire surface of the layer 11 has been covered.

In the example of FIG. 1, the layer 11 of spin cross-over material allows at least part of the first and second illuminations to propagate through it and reflect on an interface 12c of the SCO 11 facing the target surface 12. The interface 12c is for example an interior surface of the SCO 11 on the side facing the target surface 12. The reflection of these illuminations results respectively in a first and second target return light 14r, 34r, which contain information for example linked to a blood oxygen level of the target surface 12, in the case that the target surface is the skin of a human or animal body.

In the example of FIG. 1, the temperature detection system 10 further comprises at least one first light receiver 14, which is configured to capture first and second return light 14b, 34b coming from the layer of spin cross-over material 11. In other words, the first and second illuminations 13a, 33a illuminate the SCO layer 11, which in reaction generates first and second return light 14b, 34b. The first and second return light 14b, 34b contain characteristics, for example the optical intensity reflected by the SCO layer 11, which are directly representative, i.e. proportional, to the reflectivity and/or color of the SCO layer 11.

The first light receiver 14 captures the first and second return light 14b, 34b, and generates a first signal S1 based on the first return light 14b and generates a second signal S2 based on the second return light 34b. In an example, the first and second signals S1, S2 are proportional to the optical intensity of the first and second return light 14b, 34b.

The first light receiver 14 may also capture the first and target second return light 14r, 34r.

The reflectivity or color of the SCO layer 11 as a function of temperature for example are known to those skilled in the art or can be determined. For example, the reflectivity or the color of the SCO is known to change rapidly as a function of the temperature of the SCO due to an electron inner rearrangement.

In some embodiments, one or more reference values (REF) representing the behavior of reflectivity and/or color of the SCO layer 11 as a function of the temperature, is stored in a memory of the temperature detection system 10.

The analyzer module 15 for example uses the first and second signals S1, S2, and in some cases the first reference REF, in order to determine a temperature of the layer of spin cross-over material 11.

The intensity I response of the SCO as a function of the absolute temperature T may be approximated by the following expression (equation 1):

$$\text{I} \left(\text{T}\right) = \text{δ} + \text{ρ} Tanh \left(\frac{\text{T} - {\text{T}}_{\text{ref}}}{\text{σ}}\right)$$

where T_{ref} is a temperature of reference and the other parameters are constants, which in one example have the following values: δ=ρ=0.5 and σ=11.11.

In order to implement a fast and low-cost calculation, it is possible to assimilate the tanh function by a tan function for real intensities. Tan function can be calculated by the following equation (equation 2):

$$Tan \left(\text{x}\right) = {\sum }_{\text{k=1}}^{\text{N}} \frac{2 \text{x}}{{π}^{2} {\left(\text{k} - \frac{1}{2}\right)}^{2} - {\text{x}}^{2}} = \frac{\text{x}}{1 + \frac{{\text{x}}^{2}}{3 + \frac{{\text{x}}^{2}}{5 + ⋯}}}$$

As an example, a measured optical intensity of the SCO layer 11 corresponds to a given temperature of the SCO. It is therefore possible to determine the temperature, in a precise manner, remotely, at a low cost, and with a high processing speed. In another example, a measured color of the SCO layer 11 corresponds to a given temperature of the SCO. Since the temperature of the SCO layer 11 and of the target surface 12 are assumed to be equal, by determining the temperature of the SCO layer 11, the temperature of the target surface can be measured.

By maintaining the SCO at a temperature range of between -196° C. and +100° C., the SCO will not be altered by the temperature fluctuations and the inner electronical conformations, which are related.

The first correlator 17 of the computation circuit of the temperature detection system 10 of Figure is for example configured to calculate a first correlation between the first and second signals S1, S2, during the first period of time Ta. In this example, the analyzer module 15 is for example arranged to determine the temperature of the SCO 11 using the first correlation and in some cases the first reference REF.

Such an implementation allows a precise determination of the intensities and therefore of the temperature of the SCO and in fine of the target surface 12.

In an example, the calculation of the first correlation for example comprises a calculation of an autocorrelation function and/or a cross-correlation function between the first and second signals.

The auto-correlation function is for example normalized as follows (equation 3):

$$n A {C}_{{I}_{{S}_{1}} {I}_{{S}_{1}}} \left(τ\right) = \frac{{\int }_{-}^{+} {I}_{{S}_{1}} \left(t\right) {I}_{{S}_{1}} \left(t + τ\right) d t}{\sqrt{{\int }_{-}^{+} {\left|{I}_{{S}_{1}} \left(t\right)\right|}^{2} d t {\int }_{-}^{+} {\left|{I}_{{S}_{1}} \left(t\right)\right|}^{2} d t}}$$

where τ is the decay between each measurement, and I_{S1} the intensity as materialized by the first return light signal S1.

The cross-correlation function is for example normalized as follows (equation 4):

$$n C {C}_{{I}_{{S}_{1}} {I}_{S 2}} \left(τ\right) = \frac{{\int }_{-}^{+} {I}_{{S}_{1}} \left(t\right) {I}_{{S}_{2}} \left(t + τ\right) d t}{\sqrt{{\int }_{-}^{+} {\left|{I}_{{S}_{1}} \left(t\right)\right|}^{2} d t {\int }_{-}^{+} {\left|{I}_{{S}_{2}} \left(t\right)\right|}^{2} d t}}$$

where I_{S2} the intensity as materialized by the second return light signal S2.

The different operations may be performed in real time using a sliding fast Fourier transform (SFFT) by performing a calculation based on the following equation (equation 5):

$$\text{I} \left(t , k\right) = {\sum }_{n = 0}^{\text{N-1}} \text{i} \left(n - t\right) \text{w} \left(n\right) E x p \left(- \frac{j 2 π \text{} n k}{N}\right)$$

where w(n) is a sliding window function (e.g., Gaussian).

The cross-correlation function is extracted accounting for its temperature dependence for different values of the slope parameter, σ exhibiting a Gaussian behavior.

It has been found that the correlation function follows the Cardinal-Sine (SinC(x)) law with a maximum occurring when τ=0. In the vicinity of the origin, the Sine term can be approximated by its Taylor expansion as in the following expression (equation 6):

$$S i n C \left(τ\right) = \frac{Sin \left(τ\right)}{τ} = {\sum }_{n = 1}^{n = ∞} {\left(- 1\right)}^{n} \frac{{τ}^{2 n}}{\left(2 n + 1\right) !}$$

A simplified implementation is therefore possible in a portable equipment with a relatively fast processing and low consumption.

FIG. 2 illustrates the optical intensity of return light as a function of the temperature of the SCO layer 11 as known by the person of the art and represented by the one or more first references REF. The optical intensities are indicated for example by the first and second signals S1, S2. Once the correlation calculation has been applied to these signals, the intensities are for example fitted using the previous equations with the REF curves. It is then possible to determine the temperature of the SCO layer 11, and thus of the target surface.

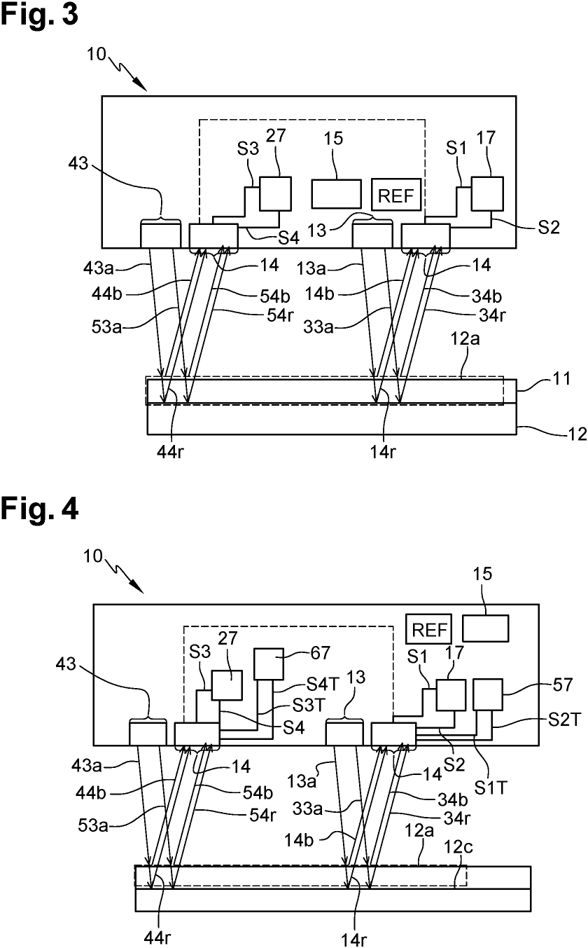

FIG. 3 illustrates schematically illustrates a temperature detection device according to an embodiment of the present disclosure.

In addition to the features illustrated in FIG. 1, the temperature detection system 10 of FIG. 3 further comprises at least one second light source 43. The second light source 43 is configured to provide third and fourth illuminations 43a, 53a of at least the first portion 12a of the SCO 11.

In an example, the second light source 43 may provide the third and fourth illuminations sequentially on several different portions of the SCO until the entirety of the SCO 11 has been covered.

In an example, the second light source 43 is formed by one or more light emitting diodes.

The third and fourth illuminations 43a, 53a are formed of light have a same second wavelength, which is for example different to the first wavelength. In other words, the third and fourth illuminations for example have the same wavelength, which is for example different from the first wavelength. The third and fourth illuminations 43a, 53a may be generated, in an example, for a period of time equal to the first period of time Ta. The third and fourth illuminations 43a, 53a may be generated sequentially and repeated in time.

In some embodiments the third and/or fourth illuminations 43a, 53a illuminate the SCO layer 11 with illumination pulses. In equivalent terms, the second light source 43 is configured to generate the third or fourth illuminations 43a, 53a in the form of pulses. This configuration may be employed in order to obtain time-of-flight information to be able to determine a distance of one or more points on the layer 11 of spin cross-over material.

The first light receiver 14 is, in the example of FIG. 3, further configured to capture third and fourth return light 44b, 54b coming from the SCO 11 and resulting respectively from the third and fourth illumination 43a, 53a. The third and fourth return light may correspond to a reflection on the SCO layer 11.

The first light receiver 14 may also capture third and fourth return light 44r, 54r resulting from the third and fourth illuminations, which propagate through the SCO layer 11 and reflect on the interface 12c of the SCO 11 facing the target surface 12.

In FIG. 3, the first light receiver 14 is represented for clarity reasons in two parts, but it can be formed by one part and formed for example by a single or a plurality of light emitting diodes.

The first light receiver 14 is, in the example of FIG. 3, further configured to generate a third signal S3 based on the third return light 44b.

The first light receiver 14 is, in the example of FIG. 3, further configured to generate a fourth signal S4 based on the fourth return light 54b. The third and fourth signals S3, S4 are generated according to the optical intensity of respectively the third and fourth return light 44b, 54b. In other terms, the intensity of the third and fourth signals S3, S4 is proportional to the optical intensity reflected by the SCO layer 11 of the respective incoming illumination.

In the example of FIG. 3, the analyzer module 15 is arranged to determine, based on the first, second, third, and fourth signals S1, S2, S3, S4, and in some cases on the first reference REF, a temperature of the layer of spin cross-over material 11. In an example the optical intensities represented by the first, second, third, and fourth signals S1, S2, S3, S4, are compared or averaged. It is then possible, as explained above in relation with FIG. 1, by fitting the obtained intensities with the first reference curves, to determine precisely the temperature of the SCO layer 11. The addition of the third and fourth illuminations for example provides a more robust and precise temperature measurement of the SCO layer 11.

The computation circuit of the temperature detection system 10 of FIG. 3 for example further comprises a second correlator 27 configured to determine a second correlation between the third and fourth signals S3, S4. The second correlation is for example calculated for a period of time of the third and fourth signals equal to the first period of time Ta.

The calculation of the second correlation for example comprises an autocorrelation calculation and/or a cross-correlation calculation, which are similar to those described above in relation with FIG. 1.

In the example of FIG. 3, the analyzer module 15 is for example configured to determine the temperature of the layer 11 of spin cross-over material based on the first and second correlations, and in some cases based on the first reference REF. The first and second correlations may be averaged to increase robustness and precision.

FIG. 4 illustrates an example of the temperature detection system 10 that can be used to further measure a blood oxygen level at the target surface 12.

The temperature detection system 10 of FIG. 4 is similar to the one described in FIG. 3 but the second light source 43 and related components are optional.

In the example of FIG. 4, the first light receiver 14 is further configured to generate a first target signal S1T based on the first target return light 14r and generate a second signal S2T based on the second target return light 34r. Optionally, the first light receiver 14 is further configured to generate a third target signal S3T based on the third target return light 44r and generate a fourth signal S4T based on the fourth target return light 54r. The first, second, third and fourth target return signals S1T, S2T, S3T, S4T are for example generated according to the optical intensity of respectively the first, second, third and fourth target return light.

In the example of FIG. 4, the temperature detection system 10 optionally comprises a fifth correlator 57 configured to determine a fifth correlation between the first and second target return signals S1T, S2T. The calculation of the fifth correlation is for example similar to the one described above in relation with FIG. 1. In an example, the fifth correlation may be calculated for a period of time of the first and second target return signals equal to the first period of time Ta and repeated in time.

In the example of FIG. 4, the temperature detection system 10 optionally comprises a sixth correlator 67 configured to determine a sixth correlation, between the third and fourth target return signals S3T, S4T. The calculation of the sixth correlation for example similar to the one described above in relation with FIG. 1. In an example, the sixth correlation may be calculated for a period of time of the third and fourth target return signals equal to the first period of time Ta and repeated in the time.

In the example of FIG. 4, the analyzer module 15 is for example configured to determine, based on at least one of the first, second, third and fourth target return signals S1T, S2T, S3T, S4T and on a Stern-Volmer constant, a blood oxygen level at the target surface 12, for example in the case where the target surface 12 is the skin of a human or animal body.

In the example of FIG. 4, the analyzer module 15 is for example additionally arranged to determine, based on the fifth and sixth correlations, and on the Stern-Volmer constant, a further blood oxygen level of the target surface 12.

In order to measure the blood oxygen level, it is possible to use the intensities determined via at least one of the first, second, third and fourth target return signals S1T, S2T, S3T, S4T and/or the fifth and/or sixth correlations, based for example on the following relations (equation 7):

$$\frac{{\text{I}}_{0}}{\text{I}} = \frac{{τ}_{{I}_{0}}}{{τ}_{I}} = 1 + {K}_{S V} . \left[{O}_{2}\right]$$

which leads to:

$$\left[{O}_{2}\right] = \frac{1}{{K}_{S V}} \left(\frac{{\text{I}}_{0}}{\text{I}} - 1\right) = \frac{1}{{K}_{S V}} \left(\frac{{τ}_{{I}_{0}}}{{τ}_{I}} - 1\right)$$

where ^{τ}Iₒ et τ_{I} are lifetimes of illuminated red blood cells in the presence and absence of oxygen, [O₂] is the blood oxygen level, I the intensity as obtained from the fifth and/or sixth correlations and/or based on at least one of the first, second, third and fourth target return signals S1T, S2T, S3T, S4T, I₀ is the intensity without the presence of oxygen and can be calibrated, and Kₛᵥ is the Stern-Volmer constant. In an example, τ_{I0} =0.86 ms ± 0.033 ms and Kₛᵥ = 0.255 ms ± 0.005 ms⁻¹.Torr⁻¹.

Optionally, in addition, cross-entropy metrics are used for evaluating the accuracy of the stochastic measurements based on the following relations (equation 8):

$$\text{Cross- Entropy} = \text{-} {\sum }_{\text{u=0}}^{N} {\sum }_{\text{v=0}}^{\text{M}} \text{I} u , v log \left(P u , v\right)$$

where, Iu,v denotes the true value i.e. 1 if sample u belongs to class v, and 0 otherwise, and Pu,v is the probability predicted of for sample u belonging to class v.

The cross-correlation functions are for example expressed as follows (equation 9):

$${\text{CC}}_{{\text{I}}_{{\text{S}}_{\text{1}}} {\text{I}}_{{\text{S}}_{\text{2}}}} = \frac{\text{Cov} \left({\text{I}}_{{\text{S}}_{1}} {\text{I}}_{{\text{S}}_{2}}\right)}{\text{σ} \left({\text{I}}_{{\text{S}}_{1}}\right) \text{σ} \left({\text{I}}_{{\text{S}}_{2}}\right)}$$

With (equation 10):

$$\text{Cov} \left({\text{I}}_{{\text{S}}_{1}} {\text{I}}_{{\text{S}}_{2}}\right) = \text{E} \left[\left({\text{I}}_{{\text{S}}_{1}} - μ \text{} 1\right) \left({\text{I}}_{2} - μ \text{} 2\right)\right]$$

where µi and σ(I_{Sᵢ}) are the expectation and standard deviation of I_{Sᵢ}. Here

$${\text{CC}}_{{I}_{{S}_{1}} {I}_{{S}_{2}}}$$

denotes a coefficient number in the interval [-1, +1]. The boundaries -1 and +1 will be reached if and only if I_{S₁} and I_{S₂} are indeed linearly related. The greater the absolute value of

$${\text{CC}}_{{I}_{{S}_{1}} {I}_{{S}_{2}}} ,$$

the stronger the dependence between I_{S₁} and I_{S₂}

The Tanh(x) presence in equation 1 may be useful in an artificial intelligence (A.I) analysis of the intensities and to machine learning (ML) algorithms, which has a similar structure and uses Sigmoid Activation Functions (SAF) as expressed here (equation 11):

$$\text{Sigmoid} \left(\text{x}\right) = \frac{{\text{e}}^{\text{x}}}{1 + {\text{e}}^{\text{x}}} = \frac{1 + \text{Tanh} \left(\text{x}\right)}{2}$$

In an example, the parameters δ and ρ are then taken to be equal to 0.5 in order to provide a relatively easy analytical transfer function for bridging Sigmoid-based representation of equation 10 and hyperbolic-tangent numerical expansion as described previously in equations 1 and 2.

In this example, an artificial neural network architecture and ML may be used for accurate extraction of a blood oxygen level of the target surface when the target surface 12 is a human or animal body surface. Each neuron transfer-function of the artificial neural network architecture is for example implemented by the following equation (equation 12):

$$\text{Output} = \text{Sigmoid} \left({\sum }_{\text{u=1}}^{\text{Q}} \left[{\text{w}}_{\text{u}} {\text{i}}_{\text{u}} + \text{b}\right]\right)$$

where wᵤ are the weighting parameters, b is a bias, iᵤ are the inputs and Sigmoid () is the Sigmoid activation function.

The artificial neural network architecture for example comprises an input layer comprising a plurality of neurons, one or more hidden layers each comprising a further plurality of neurons, and an output layer comprising a further plurality of neurons that predicts the blood oxygen level. Those skilled in the art will understand will be capable of training the artificial neural network to obtain an appropriate accuracy of the blood oxygen level at the target surface 12.

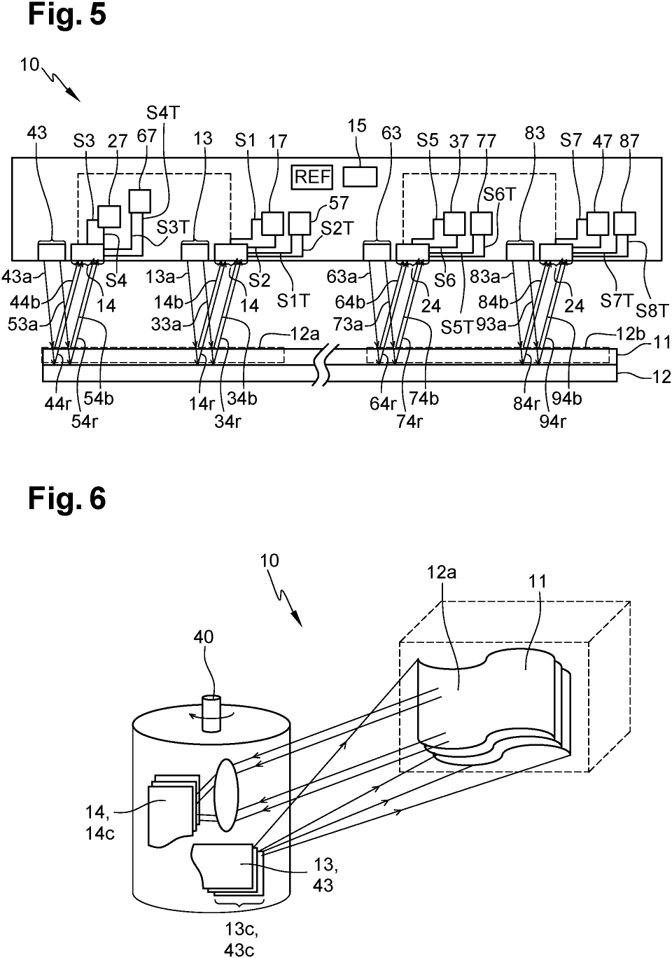

FIG. 5 schematically illustrates a temperature detection device according to a further example embodiment. FIG. 5 is similar to FIG. 4, but with all of the elements duplicated except the analyzer 15 and the first reference REF.

In the example of FIG. 5, the temperature detection system 10 comprises at least one third light source 63 configured to provide fifth and sixth illuminations 63a, 73a of at least a second portion 12b of the SCO layer 11. The fifth and sixth illuminations 63a, 73a are for example formed of light having a same third wavelength substantially equal for example to the first wavelength. In an example, the third light source 63 is for example configured to provide the third and fourth illuminations sequentially on additional portions of the SCO layer 11 so that up to the entirety of the SCO layer 11 is covered. This for example permits a temperature map of the target surface 12 to be generated.

The temperature detection system 10 of FIG. 5 for example comprises at least one fourth light source 83 configured to provide seventh and eighth illuminations 83a, 93a of at least the second portion 12b of the layer of the SCO layer 11. The seventh and eighth illuminations 83a, 93a are for example formed by light of a same fourth wavelength substantially equal for example to the second wavelength.

In an example, the fourth light source 83 may provide the seventh and eighth illuminations sequentially to additional portions of the SCO layer 11 such that up to the entirety of the SCO 11 is covered. This for example allows a temperature map of the target surface 12 to be generated.

The temperature detection system 10 of FIG. 5 for example comprises a second light receiver 24 configured to capture fifth and/or sixth and/or seventh and/or eighth return light 64b, 74b, 84b, 94b coming from the SCO layer 11 and resulting respectively from the fifth, sixth and seventh and eighth illuminations.

The second light receiver 24 is for example formed, in an example, by the first light receiver 14 and in another example, formed by at least one of the light sources 13, 43, 63, 83. In other words the lights sources and the light receivers may be formed by a one and unique photodiode.

In an example, the first light source 13 and/or the second light source 43 and/or the third light source 63 and/or the fourth light source 83 are mobile in relation to the layer of spin cross-over material 11.

The second light receiver 24 is for example configured to generate a fifth signal S5 based on the fifth return light 64b. The second first light receiver 24 is for example configured to generate a sixth signal S6 based on the sixth return light 74b. The second light receiver 24 is for example configured to generate a seventh signal S7 based on the seventh return light 84b. The second light receiver 24 is for example configured to generate an eighth signal S8 based on the eighth return light 94b. The fifth, sixth, seventh and eighth signals S5, S6, S7, S8 are for example generated according to the optical intensity of respectively the fifth, sixth, seventh and eighth return lights 64b, 74b, 84b, 94b.

In the example of FIG. 5, the temperature detection system 10 comprises a third correlator 37 configured to calculate a third correlation between the fifth and sixth signals S5, S6. The third correlation is for example calculated based on an autocorrelation calculation and/or based on a cross-correlation calculation similar to ones described previously in relation with FIG. 1.

The temperature detection system 10 also for example comprises a fourth correlator 47 configured to calculate a fourth correlation between the seventh and eighth signals S7, S8. The fourth correlation is for example calculated based on an autocorrelation calculation and/or based on a cross-correlation calculation similar to the ones described previously in relation with FIG. 1.

In the example of FIG. 5, the analyzer module 15 is for example configured to determine the temperature of the SCO layer 11 at the second portion 12b using at least one of the fifth, sixth, seventh and eighth signals S5, S6, S7, S8 and/or the third and fourth correlations, and in some cases the first reference REF as described for example in the paragraph related to FIG. 1.

The example of FIG. 5 allows to speed up the process of temperature detection on a larger scale target surface 12 such as a DUT.

Optionally, the example of FIG. 5 is compatible with a blood oxygen level determination on different portions 12a, 12b. The blood oxygen level determination, for each portion 12a, 12b, may be achieved separately as described above.

In the example of FIG. 5, the second light receiver 24 is for example further configured to generate a fifth target signal S5T based on the fifth target return light 64r and optionally to generate a sixth signal S6T based on the fifth target return light 74r. Optionally, the second light receiver 24 is further configured to generate a seventh target signal S7T based on the seventh target return light 84r and generate a eighth signal S8T based on the eighth target return light 94r. The fifth, sixth, seventh and eighth target return signals S5T, S6T, S7T, S8T are for example generated according to the optical intensity, otherwise said the luminescence, as comprised in respectively the fifth, sixth, seventh and eighth target return lights. The blood oxygen level determination can for example be made in a similar manner to what is described above in relation with FIG. 4.

The blood oxygen level is for example determined by using the intensities measurements in the first and second portions 12a, 12b based on the following equation (equation 13) :

$$\frac{\text{I}}{{\text{I}}_{0}} = \left(\frac{{\text{f}}_{1}}{1 + {\text{K}}_{{\text{SV}}_{1}} . \left[{\text{O}}_{2}\right]} + \frac{{\text{f}}_{1}}{1 + {\text{K}}_{{\text{SV}}_{2}} . \left[{\text{O}}_{2}\right]}\right)$$

where I₀ and I are, respectively, the luminescence/optical intensities in the absence and presence of oxygen, f1, and f2 = 1 - f1, are the fractions of the total emission/luminescence for each component under unquenched conditions, and K_{SV1} and K_{SV2} are the associated Stern-Volmer constants for each portion 12a and 12b. In general, it is assumed f1 + f2 = 1, so f1 = f and f2 = 1 - f.

FIG. 6 illustrates schematically an example embodiment of the detection system 10 according to which the first light source 13, the second light source 43 and the first light receiver 14 are fixed in relation to each other and mobile around a first axis 40.

In this case, in order to realize the correlations as described above, a time compensation, for example implemented by a delay line, is introduced in order to compensate for differences in the timing of the signals from each light source due to the rotation of the various light sources.

Optionally, the first and/or the second light source 13, 43 comprise a plurality of light emitters 13c, 43c, for example 4, 6 or 8 light emitters, configured to illuminate a plurality of different portions of the SCO layer 11.

In an example, the first light source 13, the second light source 43 and the first light receiver 14 form a first LIDAR.

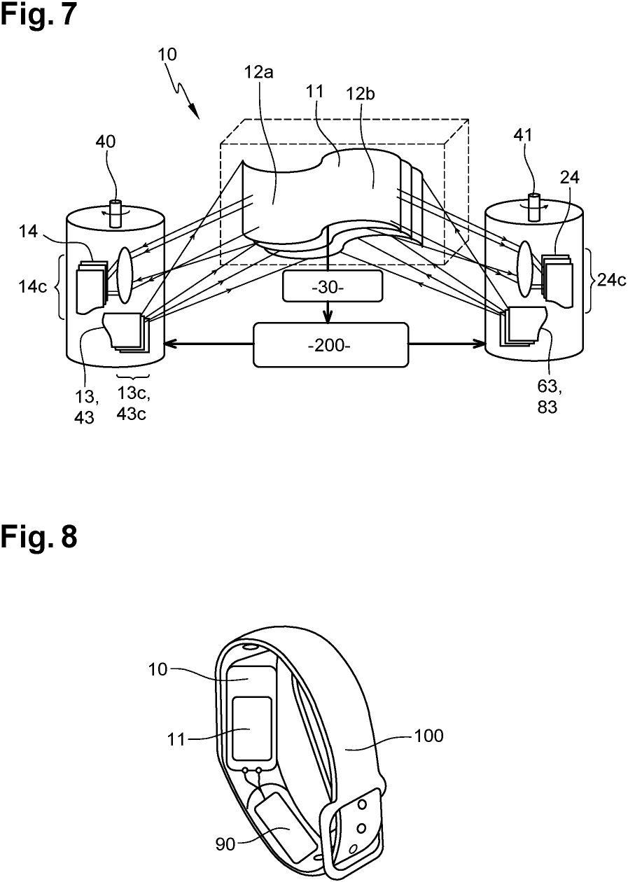

FIG. 7 illustrates an embodiment of the disclosure similar to the example of FIGS. 5 and 6, but in which the third light source 63 and the fourth light source 83 and the second light receiver 24 are fixed in relation to each other and mobile around a second axis 41.

The first or second light receivers 14, 24 for example comprise a plurality of light detectors 14c, 24c, for example 4, 6 or 8 light detectors, configured to receive return light from a plurality of different portions of the layer 11 of spin cross-over material. In an example, the plurality of light emitters and/or the plurality of light detectors are arranged in a linear array or a 2-dimensional matrix of pixels. This configuration for example permits a rapid mapping of temperatures, for example for a DUT in production, which should be tested for only a few seconds.

In an example, the third light source 63 and the fourth light source 83 and the second light receiver 24 form a second LIDAR. Such a configuration is for example useful for retrieving fast 3D mapping of temperatures, for example of 3D electrical components.

Correlation calculations may be performed by a computation circuit 200, in a similar manner to what is described above in relation to FIG. 1, between the signals received by the first and second light receivers 14, 24.

A calibration circuit 30 may additionally be provided in order to perform calibration prior to calculating the correlations by the computation circuit 200.

FIG. 8 illustrates a bracelet 100 comprising a temperature detection system 10 as described herein. The SCO layer 11 of the temperature detection system 10 of FIG. 8 is arranged to be in contact with the skin of the user.

In an example, the bracelet 100 comprises an energy harvesting device 90 configured to harvest heat energy from the target surface 12 to power components of the temperature detection system 10.

In an example, the target surface 12 is the skin of a user of the bracelet, for example in the wrist or ankle region, and energy is harvested based on a temperature gradient between the skin temperature and an ambient air temperature.

A such bracelet is useful for providing temperature measurements of the user and in some cases a blood oxygen level of the user.

Another advantage of the use of SCO in a temperature detection device in contact with the skin of a user, as proposed herein, is that SCO is not toxic and is compatible with the human body without harming the user.

FIG. 9 illustrates a test system according to an example embodiment of the present disclosure.

FIG. 9 illustrates in particular a DUT 122, and a layer 11 of SCO illustrated by dashed lines has been applied on the DUT 122, for example mixed with or otherwise forming a protection layer, deposited for example on the electric components, and formed for example by a nitride. The DUT 122 here is part of the temperature detection system 10 and is specifically designed to provide a current reference circuit 121. The current reference circuit 121 is calibrated, in order to provide a known current in conductive lines which are near the DUT. The temperature of the conductive lines, as determined off-line or in-line by the temperature detection system 10 with, for example, its first and second illuminations and the corresponding return light 14b, 34b, is related to the known current applied inside this current reference circuit 121. This configuration forms a correspondence table between the current and the determined temperature. In an example, the current reference circuit 121 is controlled by a control circuit, which performs a static and dynamic biasing of the current supplying the current reference circuit 121. The static and dynamic biasing is for example regulated by a bias correlator and calibration sensors as well as a signal processing using space and time correlation of the calibration signals.

Once in-line, the temperature measurement performed by the temperature detection system 10 on the DUT surface is for example directly linked to a current mapping of the DUT for example on a control screen. This configuration is useful for the recognition of high currents spots that could appear in a DUT and indicate a faulty behavior.

The various calculations of the various embodiments of the present disclosure may be performed using advanced ASIC Photonics, and the accuracy obtained for the measured temperature is for example below 0.05° C.

Various embodiments and variants have been described. Those skilled in the art will understand that certain features of these embodiments can be combined and other variants will readily occur to those skilled in the art.

Finally, the practical implementation of the embodiments and variants described herein is within the capabilities of those skilled in the art based on the functional description provided hereinabove.

1. A temperature detection system comprising:
a layer of spin cross-over material in thermal contact with a target surface;
at least one first light source configured to provide a first and a second illumination of at least a first portion of the layer of spin cross-over material (44);
at least one first light receiver configured to:
capture first and second return light coming from the layer of spin cross-over material and resulting respectively from the first and second illuminations;
generate a first signal based on the first return light; and
generate a second signal based on the second return light; and
a computation circuit configured to determine, based at least on a correlation between the first and second signals, a temperature of the layer of spin cross-over material.

2. The temperature detection system of claim 1, wherein the first and second signals are generated according to a first characteristic of respectively the first and second return lights which is relative to at least one of a reflectivity or a color of the layer of spin cross-over material.

3. The temperature detection system of claim 1, wherein the first characteristic is an optical intensity.

4. The temperature detection system of claim 1, wherein the layer of spin cross-over material comprises [Fe(HB(1,2,4-triazol-1-yl)₃)₂]bis[hydrotris(1,2,4-triazol-1-yl)borate]Fe(II).

5. The temperature detection system of claim 1, wherein a layer of a temperature conductive material is arranged between the target surface and the layer of spin cross-over material.

6. The temperature detection system of claim 1, wherein the first and second illuminations are each formed of light having a same first wavelength.

7. The temperature detection system of claim 1, wherein the computation circuit is configured to determine the correlation between the first and second signals using an auto-correlation calculation or a cross-correlation calculation, the auto-correlation calculation being performed when the first and second signals are issued from a same portion of the layer of spin cross-over material and the cross-correlation calculation being performed when the first and second signals are issued from different portions of the layer of spin cross-over material, the different portions being adjacent portions, the auto-correlation or cross-correlation calculation being approximated by a Cardinal-Sine function.

8. The temperature detection system of claim 1, wherein the computation circuit is configured to determine the temperature of the layer of spin cross-over material further based on one or more reference values associated with known temperatures.

9. The temperature detection system of claim 1, further comprising at least one second light source configured to provide a third and a fourth illumination of at least the first portion of the layer of spin cross-over material, wherein the first and second illuminations are each formed of light having a same first wavelength and the third and fourth illuminations are each formed of light having a same second wavelength different to the first wavelength;
the at least one first light receiver being configured to:
capture third and fourth return light coming from the layer of spin cross-over material and resulting respectively from the third and fourth illuminations;
generate a third signal based on the third return light; and
generate a fourth signal based on the fourth return light;
the third and fourth signals being generated according to an optical intensity of respectively the third and fourth return lights; and
the computation circuit being configured to determine the temperature of the layer of spin cross-over material further based on a correlation between the third and fourth signals.

10. The temperature detection system of claim 9, further comprising:
at least one third light source configured to provide a fifth and sixth illuminations of at least a second portion of the layer of spin cross-over material, the fifth and sixth illuminations being formed of light having a third wavelength substantially equal to the first wavelength;
at least one fourth light source configured to provide a seventh and eighth illuminations of at least the second portion of the layer of spin cross-over material, the seventh and eighth illuminations being formed of light having a fourth wavelength substantially equal to the second wavelength;
a second first light receiver being configured to:
capture fifth, sixth, seventh and eighth return light coming from the layer of spin cross-over material and resulting respectively from the fifth, sixth and seventh and eighth illuminations;
generate a fifth signal based on the fifth return light;
generate a sixth signal based on the sixth return light;
generate a seventh signal based on the seventh return light; and
generate an eighth signal based on the eighth return light;
the fifth, sixth, seventh and eighth signals being generated according to an optical intensity of respectively the fifth, sixth, seventh and eighth return light; and
the temperature detection system comprising a third correlator configured to run a third operation of correlation, between the fifth and sixth signals;
the third operation of correlation comprising at least one of an autocorrelation operation or a cross-correlation operation;
the temperature detection system comprising a fourth correlator configured to run a fourth operation of correlation, between the seventh and eighth signals;
the fourth operation of correlation comprising at least one of an autocorrelation operation or a cross-correlation operation; and
an analyzer being arranged to determine the temperature of the layer of spin cross-over material at the second portion using a result of the third and fourth operations of correlation and the first reference (REF).

11. The temperature detection system of claim 10, the at least one first light source and the at least one second light source are each configured to provide the first, second, third, and fourth illuminations to illuminate sequentially, portions of the layer of spin cross-over material;
the at least one first light receiver being configured to:
generate the first signal for each portion of the layer of spin cross-over material sequentially illuminated by the first illumination;
generate the second signal for each portion of the layer of spin cross-over material sequentially illuminated by the second illumination;
generate the third signal for each portion of the layer of spin cross-over material sequentially illuminated by the third illumination; and
generate the fourth signal for each portion of the layer of spin cross-over material sequentially illuminated by the fourth illumination;
at least one second light receiver being configured to:
generate the fifth signal for each portion of the layer of spin cross-over material sequentially illuminated by the fifth illumination;
generate the sixth signal for each portion of the layer of spin cross-over material sequentially illuminated by the sixth illumination;
generate the seventh signal for each portion of the layer of spin cross-over material sequentially illuminated by the seventh illumination; and
generate the eighth signal for each portion of the layer of spin cross-over material sequentially illuminated by the eighth illumination; and
the first, second, third, fourth, fifth, sixth, seventh and eighth signals being generated according to the optical intensity of the respective return lights.

12. The temperature detection system of claim 11, wherein:
at least one of the first light source, the second light source, the third light source, or the fourth light source are mobile in relation to the layer of spin cross-over material;
the at least one first light source, the at least one second light source, and the at least one first light receiver are fixed in relation to each other and mobile around a first axis; and
the at least one third light source, the at least one fourth light source, and the at least one second light receiver are fixed in relation to each other and mobile around a second axis.

13. (canceled)

14. The temperature detection system of claim 10, wherein:
at least one of the at least one first light source or the at least one second light source comprises a plurality of light emitters configured to illuminate a plurality of different portions of the layer of spin cross-over material;
at least one of the at least one first light receiver or the at least one second light receiver comprises a plurality of light detectors configured to receive return light from a plurality of different portions of the layer of spin cross-over material; and
at least one of the plurality of light emitters or the plurality of light detectors are arranged in a linear or 2-dimentional matrix of pixels.

15. (canceled)

16. The temperature detection system of claim 9, wherein:
the layer of spin cross-over material is arranged to allow at least part of the first, second, third, and fourth illuminations to propagate through it and reflects on an interface of the layer of spin cross-over material facing the target surface resulting respectively in a first, second, third, and fourth target return light;
the first light receiver being further configured to:
generate a first target signal based on the first target return light;
generate a second target signal based on the second target return light;
generate a third target signal based on the third target return light; and
generate a fourth target signal based on the fourth target return light;
the first, second, third, and fourth target signals being generated according to an optical intensity of respectively the first, second, third, and fourth target return light;
wherein the computation circuit is further configured to:
determine a correlation between the first and second target return signals; and
determine a correlation between the third and fourth target return signals; and
the computation circuit being capable of determining, based on the correlations between the first and second target return signals and between the third and fourth target return signals and on a Stern-Volmer constant, a blood oxygen level at the target surface.

17. The temperature detection system of claim 1, wherein the target surface is a surface of a device under test, the device under test comprising one or more transistors or one or more integrated circuit chips, a surface of a vehicle, a surface of an animal or a surface of a human body.

18. The temperature detection system of claim 17, further comprising an energy harvesting device configured to harvest heat energy from the target surface to power components of the temperature detection system.

19. The temperature detection system of claim 1, wherein the at least one first light source and the at least one first light receiver are formed together in a same photodiode.

20. A temperature detection device comprising the temperature detection system of claim 1.

21. A bracelet comprising the temperature detection system of claim 1.

22. The bracelet of claim 21, further comprising an energy harvesting device, wherein the target surface is the skin of a user of the bracelet and energy is harvested based on a temperature gradient between the skin temperature and an ambient air temperature.
